(19) United States
(12) Patent Application Publication
(10) Pub. No.: US 2017/0080087 A1
(43) Pub. Date: Mar. 23, 2017

(54) Combination Therapies for Treating Her2-Positive Cancers that are Resistant to Her2-Targeted Therapies

(71) Applicant: MERRIMACK PHARMACEUTICALS, INC., CAMBRIDGE, MA (US)

(72) Inventors: CHRISTOPHER ESPELIN, BELMONT, MA (US); ELENA GERETTI, CAMBRIDGE, MA (US); VICTOR MOYO, RINGOES, NJ (US); JOSEPH G. REYNOLDS, NORTH ANDOVER, MA (US); THOMAS WICKHAM, GROTON, MA (US); BART S. HENDRIKS, BELMONT, MA (US)

(73) Assignee: MERRIMACK PHARMACEUTICALS, INC., CAMBRIDGE, MA (US)

(21) Appl. No.: 15/369,094

(22) Filed: Dec. 5, 2016

Related U.S. Application Data
(63) Continuation of application No. 14/819,317, filed on Aug. 5, 2015.
(60) Provisional application No. 62/033,423, filed on Aug. 5, 2014.

(51) Int. Cl. A61K 39/395; A61K 31/704; A61K 45/06; C07K 16/28; C07K 16/32; A61K 9/127; A61K 9/00

(52) U.S. Cl. CPC A61K 39/39558; A61K 9/127; A61K 31/704; A61K 9/0019; C07K 16/2863; C07K 16/32; A61K 47/48823; A61K 47/48384; A61K 47/48569; A61K 47/48615; A61K 47/48584; A61K 45/06; C07K 2317/24; C07K 2317/622; A61K 2039/545

(57) ABSTRACT

Methods for treating cancer patients (e.g., cancer patients resistant or intolerant to pertuzumab and ado-trastuzumab emtansine) having HER2-positive tumors are disclosed. The methods comprise administering to a patient a therapeutically effective amount of a combination of a doxorubicin-loaded immunoliposome with a targeting moiety that is an anti-HER2 antibody that is not an inhibitor of HER2 signaling and an anti-cancer therapeutic comprising a doxorubicin-free anti-cancer therapeutic comprising a different anti-HER2 antibody.

### CROSS-REFERENCE TO RELATED APPLICATION

This application claims priority to U.S. Provisional Application Ser. No. 62/033,423, filed Aug. 5, 2014, the disclosure of which is hereby incorporated by reference in its entirety.

### BACKGROUND

Over-expression of Human Epidermal Growth Factor Receptor 2 (HER2) is associated with a variety of cancers including, e.g., breast cancer, ovarian cancer, stomach cancer, uterine cancer, melanoma, and cholangiocarcinoma. In many cases HER2 over-expression is associated with aggressive, metastatic forms of breast cancer that have high rates of recurrence and/or are associated with poor patient prognosis.

Anthracyclines such as doxorubicin have been used as effective cancer therapies for decades, and anthracycline-based regimens have demonstrated clinical benefit for treating breast cancer. Unfortunately, such anthracycline-based regimens are associated with significant toxicities which have limited their therapeutic use, such as, for example, cardiotoxicity associated with acute and chronic cardiac dysfunction. In an effort to improve the safety and efficacy of currently available anthracyclines, immunoliposomal formulations have been prepared that contain the anthracycline doxorubicin in liposomes having antibodies in their exterior surfaces that target HER2 overexpressing cancer cells and do not block (i.e., the antibodies do not inhibit) HER2-mediated intracellular signal transduction (“HER2 signaling”).

Another approach to treating HER2 overexpressing cancers has focused on the use of anti-HER2 antibodies that inhibit HER2 signaling, which is believed to drive cell proliferation and other oncogenic characteristics of malignant tumor cells. For example, trastuzumab (HERCEPTIN®), pertuzumab (PERJETA®) and ado-trastuzumab emtansine (KADCYLA®, also referred to as T-DM1) comprise therapeutic anti-HER2 antibodies that block HER2 signaling and are widely used to treat HER2 overexpressing tumors. While such HER2 signaling-inhibitory anti-HER2 antibody therapeutics are often highly effective, many treated tumors become resistant to these therapies, and some patients are or become drug intolerant (i.e., intolerant to these drugs).

There is an unmet need for new therapeutic approaches for effective treatment of patients who become resistant (as evidenced, e.g., by tumor progression following treatment) or are, or become intolerant to, treatment with anti-HER2 antibodies that inhibit HER2 signaling. The following disclosure provides methods and compositions that address these needs and provide additional benefits.

### SUMMARY

Provided herein are methods and compositions for follow-on treatment of a HER2-positive cancer (e.g., a HER2 positive tumor) in a human patient, the methods comprising co-administering to the patient 1) a preparation of an immunoliposome, such as MM-302, which comprises encapsulated doxorubicin and a an immunoliposome-associated anti-HER2 antibody that is not an inhibitor of HER2 signaling and is exteriorly oriented on the immunoliposome so as to be able to bind to an antigen that is external to the immunoliposome and 2) a doxorubicin-free anti-cancer therapeutic, e.g., a preparation comprising an anti-HER2 antibody that; a) is an inhibitor of HER2 signaling, or that b) does not bind to the same epitope of HER2 that is bound by the immunoliposome-associated antibody, or that c) does not compete with the immunoliposome-associated antibody for immunospecific binding to HER2, or any combination of a), b), and c). The combination is co-administered (or is for co-administration), e.g., according to a clinical dosage regimen disclosed herein, i.e., particular doses (amounts) given via a particular modality (e.g., intravenous infusion over a prescribed period of time) according to a specific dosing schedule. In preferred embodiments, prior to the follow-on treatment, the patient had received treatment (in any order, simultaneous or sequential or any combination thereof) with one or more of 1) trastuzumab, 2) pertuzumab, and 3) ado-trastuzumab emtansine, and experienced tumor progression following treatment initiation with, or was intolerant to treatment with pertuzumab and experienced tumor progression following treatment initiation with, or was intolerant to treatment with ado-trastuzumab emtansine. In additional preferred embodiments, the patient has not been treated with a systemically administered anthracycline prior to receiving the follow-on treatment provided herein.

Another aspect is the use of an immunoliposome comprising encapsulated doxorubicin and a HER2-binding antibody, in combination with an anti-HER2 therapeutic that comprises trastuzumab or another anti-HER2 antibody, for the follow-on treatment of a HER2-positive tumor in a human patient by administration to the patient a therapeutically effective amount of each of the immunoliposome and the antibody, wherein, prior to the follow-on treatment, the patient had received treatment with one or more of 1) trastuzumab, 2) pertuzumab, and 3) ado-trastuzumab emtansine, and had experienced tumor progression following such prior treatment initiation with, or was intolerant to treatment with pertuzumab and had experienced tumor progression following such prior treatment initiation with, or was intolerant to such prior treatment with ado-trastuzumab emtansine.

In some embodiments, (i) the immunoliposome is an immunoliposome comprising encapsulated doxorubicin and an immunoliposome-associated anti-HER2 antibody that is not an inhibitor of HER2 signaling and is oriented on the immunoliposome so as to be able to bind to an antigen that is external to the immunoliposome and (ii) the anti-HER2 therapeutic is a doxorubicin-free therapeutic comprising an anti-HER2 antibody that a) is an inhibitor of HER2 signaling, or that b) does not bind to the same epitope of HER2 that is bound by the immunoliposome-associated antibody, or that c) does not compete with the immunoliposome-associated antibody for immunospecific binding to HER2, or that a) and b), or that a) and c), or that b) and c), or that a) and b) and c).

In various embodiments, the HER2-positive cancer is a breast cancer. The HER2-positive breast cancer may test positive for estrogen receptor and may be a HER2 non-amplified invasive breast cancer. The HER2-positive breast cancer may be advanced. The HER2-positive breast cancer may be metastatic. The HER2-positive breast cancer may be advanced/metastatic breast cancer. In other embodiments, the HER2-positive cancer is, e.g., bladder cancer, sarcoma, endometrial cancer, esophageal cancer, gastric cancer, gastro-esophageal junction cancer, ovarian cancer, lung cancer, colorectal cancer pancreatic cancer, or multiple myeloma.

In other embodiments, the immunoliposome is administered by an intravenous, intrathecal, intravesicular, intraperitoneal, or intramuscular route.

In various embodiments, the signaling-inhibitory anti-HER2 antibody comprised by the doxorubicin-free anti-cancer therapeutic is an anti-HER2 monoclonal antibody, an anti-HER2 oligoclonal antibody, or an anti-HER2 polyclonal antibody, or an anti-HER2 antibody-drug-conjugate or an anti-HER2/anti-HER3 bispecific antibody, e.g., trastuzumab, pertuzumab, ado-trastuzumab emtansine or MM-111. In other embodiments, the doxorubicin-free anti-cancer therapeutic does not comprise an antibody, and comprises one or more HER2-active tyrosine kinase inhibitor(s), e.g., lapatinib, canertinib, mubritinib, afatinib, varlitinib, and dacomitinib.

In a preferred embodiment, the immunoliposome is MM-302.

In still other embodiments, the HER2-expressing cancer is further characterized as being HER2¹⁺, HER2²⁺, HER2³⁺ (e.g., via the HERCEPTEST® assay or another such semi-quantitative immunohistochemical assay using a polyclonal anti-HER2 primary antibody), or is HER2-positive, FISH (fluorescent in-situ hybridization)-negative (for HER2 gene amplification) or is FISH-positive.

In yet another embodiment, the immunoliposome-associated anti-HER2 antibody is a single-chain Fv (scFv).

In another aspect, herein provided are compositions for and a methods of treatment of HER2-positive breast cancer in a human patient, the method including co-administering to the patient a therapeutically effective amount of MM-302 and a therapeutically effective amount of trastuzumab.

In another aspect, techniques disclosed herein provide a method for use in treating a HER2-positive cancer in a human patient including a safe and effective amount of MM-302 and a first anti-HER2 antibody, and a safe and effective amount of trastuzumab.

In another embodiment, a method is provided for treating a HER2-positive breast cancer in a human patient, the method comprising: determining a safe and effective dosage for an anthracycline-loaded anti-HER2 immunoliposome for the patient; administering the immunoliposome to the patient at the safe and effective dosage for the immunoliposome; determining a safe and effective dosage for an anti-HER2 antibody for the patient; and administering the antibody to the patient at the safe and effective dosage for the antibody; where the immunoliposome and the anti-HER2 antibody are co-administered. In various embodiments, the immunoliposome is MM-302.

In other embodiments, the signaling-inhibitory anti-HER2 antibody is formulated for intravenous administration at a dose of 2 mg/kg, 4 mg/kg, 6 mg/kg, 8 mg/kg, or 10 mg/kg.

In another embodiment, the co-administration does not cause cardiotoxicity to the patient to any greater extent than is caused by monotherapy administration of the signaling-inhibitory anti-HER2 antibody.

In another aspect, the invention provides method of treatment of a HER2-positive cancer in an anthracycline naïve human patient, the method comprising co-administering to the patient a therapeutically effective amount of each of (i) an immunoliposome comprising an encapsulated anthracycline and a targeting moiety that is a first anti-HER2 antibody and (ii) an anti-cancer therapeutic comprising a signaling-inhibitory anti-HER2 antibody.

In certain embodiments, treatment in accordance with the methods provided herein does not result in a reduction of left ventricular ejection fraction (LVEF) of greater than 10% in more than 0.5% or more than 1%, or more than 2% of treated patients. In certain embodiments, the reduction of left ventricular ejection fraction LVEF is not greater than 5%. In certain embodiments, the signaling-inhibitory anti-HER2 antibody is trastuzumab.

In another aspect, a kit is provided comprising a first container comprising: i) a second container containing a MM-302; and ii) instructions for co-administration of the MM-302 with a signaling-inhibitory anti-HER2 antibody according to any of the above-described aspects and embodiments. In certain embodiments, the first container further comprises a third container comprising at least one dose of the signaling-inhibitory anti-HER2 antibody.

In some embodiments, the immunoliposome is administered intravenously.

In another embodiment, the patient has not previously been treated with a systemically administered anthracycline.

In some embodiments, the immunoliposome-associated anti-HER2 antibody is a single-chain Fv (scFv).

In other embodiments, scFv is an F5 scFv comprising the amino acid sequence encoded by ATCC plasmid deposit designation PTA7843.

In another embodiment, the immunoliposome is MM-302 and the doxorubicin-free anti-cancer therapeutic is trastuzumab.

In some embodiments, the tumor is a breast cancer tumor.

In other embodiments, the breast cancer is histologically or cytologically characterized as invasive cancer of the breast.

In some embodiments, the breast cancer is either or both of locally advanced and metastatic.

In other embodiments, the breast cancer is not amenable to resection with curative intent.

In some embodiments, prior to initial administration of the immunoliposome, an antihistamine is administered orally or intravenously as prophylactic premedication.

In another embodiment, the use comprises at least one 3-week treatment cycle in which the signaling-inhibitory anti-HER2 antibody is administered at a dose of 6 mg/kg per administration and the immunoliposome is administered at a dose of 30 mg/m² per administration; wherein, when the at least one cycle is a single cycle, the anti-HER2 antibody and the immunoliposome are each administered once;

and wherein, when the at least one cycle is a plurality of cycles, the signaling-inhibitory anti-HER2 antibody is administered every three weeks and the immunoliposome is administered every three weeks or every four weeks.

### BRIEF DESCRIPTION OF THE DRAWINGS

FIG. 1: Exemplary flow chart for treatment of patients in accordance with methods disclosed herein. “Study treatment” refers to treatment via co-administration of MM-302 and trastuzumab, “LVEF” indicates left ventricular ejection fraction.

FIG. 2: NCI-N87 xenograft tumor study data—MM-302+trastuzumab after development of resistance to trastuzumab as described in Example 1.

FIG. 3: BT474-M3 xenograft tumor study data—MM-302+trastuzumab after development of resistance to T-DM1 (ado-trastuzumab emtansine) as described in Example 2.

FIGS. 4A-4D are a series of bar charts showing patient treatment outcomes after treatment with MM-302+trastuzumab according to Example 5. FIGS. 4A and 4C show change in patients' tumor size and FIGS. 4B and 4D show patient progression-free survival. Hatched bars indicate patients who previously received and progressed on treatment with both T-DM1 and pertuzumab and open bars represent patients who had previously been treated with T-DM1 only (FIGS. 4A and 4B) or patients who had previously been treated with pertuzumab only (FIGS. 4C and 4D).

FIG. 5A depicts graphs showing treatment BT-474-M3 cells with MM-302 alone (squares), T-DM1 alone (diamonds), and the combination of MM-302 and T-DM1 (triangles) compared to untreated cells (circles) as described in Example 6. A summary of all cell lines tested is shown in FIG. 5B.

### DETAILED DESCRIPTION

It has surprisingly been discovered that concurrent or sequential co-administration of MM-302 and an anti-HER2 antibody such as trastuzumab, can result in the safe and efficacious treatment of HER2 overexpressing cancer (e.g., breast cancer) in patients who have previously been treated (as described herein) with trastuzumab, pertuzumab, and ado-trastuzumab emtansine. Accordingly, compositions and methods are provided that, when used in accordance with this disclosure, are safe and effective for treating patients with cancer that has been histologically or cytologically confirmed positive for HER2 (i.e., HER2⁺), particularly wherein the cancer has been previously treated with one or more or all of trastuzumab, pertuzumab, and ado-trastuzumab emtansine.

### TERMINOLOGY

Immunoliposomes are antibody-(typically antibody fragment) targeted liposomes that provide advantages over liposomes that lack antibodies because, with properly selected antibodies, they are selectively internalized by cells bearing cell surface antigens targeted by the antibody. Such antibodies and immunoliposomes are described, for example, in the following U.S. patents and patent applications: U.S. Pat. Nos. 8,173,424; 7,892,554 and 7,244,826; (“Internalizing ErbB2 antibodies”) US 2010-0068255 and U.S. Pat. Nos. 6,214,388, 7,135,177, and 7,507,407 (“Immunoliposomes that optimize internalization into target cells”); U.S. Pat. No. 6,210,707 (“Methods of forming protein-linked lipidic microparticles and compositions thereof”); and U.S. Pat. No. 7,022,336 (“Methods for attaching protein to lipidic microparticles with high efficiency”). With regard to such antibodies and liposomes, the following US and international patents and patent applications describe assays, cell lines, and related technologies relevant to this disclosure: U.S. Pat. No. 7,846,440 (“Antibodies against ErbB3 and uses thereof”) and U.S. patent Ser. No. 12/757,801, PCT/US2009/040259, and PCT/US2009/60721 (“Human Serum Albumin Linkers and Conjugates Thereof”).

“Follow-on treatment” refers to treatment of a disease by administration of one or more previously un-administered therapeutic agents to a patient following the development of resistance or intolerance to one or more previously administered therapeutic agents used to treat the disease that is no longer responsive to, or can no longer be treated with (due to intolerance) the previously administered therapeutic agent. In the particular context here used, it refers to the administration of an MM-302-comprising therapeutic regimen to a patient who has become resistant or intolerant to treatment with an inhibitor of HER2 signaling, e.g., treatment comprising an anti-HER2 antibody that inhibits HER2 signaling.

“MM-302” is a unilamellar lipid bilayer vesicle of approximately 75-110 nm in diameter that encapsulates an inner aqueous space which contains doxorubicin in a gelated or precipitated state. The lipid membrane is composed of phosphatidylcholine, cholesterol, and a polyethyleneglycol-derivatized phosphatidylethanolamine in the amount of approximately one PEG molecule for 200 phospholipid molecules, of which approximately one PEG chain for each 1780 phospholipid molecules bears at its end an F5 single-chain Fv antibody fragment that is exposed on the outer surface of the vesicle and immunospecifically binds to HER2. The F5 sequence is encoded by ATCC plasmid deposit designation PTA7843. MM-302 is described (together with methods of making and using MM-302) in, e.g., co-pending PCT Patent Publication No. WO 2012/078695 (U.S. Patent Publication No: 2014-0023698, filed Jun. 6, 2013. Other relevant disclosures may be found in co-pending international application PCT/US2014/033548. The term “antibody” includes antibodies and antibody variants (including antibody fragments) comprising at least one antibody-derived antigen binding site (e.g., at least two CDRs, a VH/VL region or an Fv) that specifically bind to HER2. An antibody may be in any naturally occurring or engineered form, e.g., a human antibody, a humanized antibody, a bispecific antibody, or a chimeric antibody. An antibody may also be a Fab, Fab′2, ScFv, SMIP, Affibody®, nanobody, or a domain antibody. An antibody may also be any of the following isotypes: IgG1, IgG2, IgG3, IgG4, IgM, IgA1, IgA2, IgAsec, IgD, and IgE.

“Anthracyclines” refers to a class of drugs used in cancer chemotherapy that are structurally related to (and include) daunorubicin, which is a natural product that can be isolated from Streptomyces peucetius var. caesius. Other exemplary anthracyclines include, but are not limited to, doxorubicin, epirubicin, idarubicin, and valrubicin. Use of daunorubicin is limited due to sometimes fatal cardiotoxicity, a side effect associated to varying degrees with other anthracyclines, such as doxorubicin, as well.

As used herein, “cancer” refers to a condition characterized by abnormal, unregulated and malignant cell growth. In some embodiments, the cancer tumor is a HER2⁺ solid tumor type, e.g., a melanoma, a cholangiocarcinoma, clear cell sarcoma, or an esophageal, head and neck, endometrial, prostate, breast, ovarian, gastric, gastro-esophageal junction (GEJ), colon, colorectal, lung, bladder, pancreatic, salivary gland, liver, skin, brain, squamous cell, small-cell lung, non-small cell lung, cervical, thyroid or renal cancer.

HER2⁺ cancers are those in which tumor cells overexpress Human Epidermal Growth Factor Receptor 2 (HER2), which is also known as NEU, ErbB2, CD340, and p185. In routine clinical settings, a tumor that overexpresses HER2 is one that is identified as being HER2 “3+” or HER2 “2+” by immunohistochemistry (e.g., by HercepTest®), or, as measured by fluorescence in situ hybridization (FISH) are determined to be HER2 gene-amplified (i.e., FISH+). In some embodiments, HER2⁺ indicates the presence of at least on the order of 200,000 HER2 receptors per cell. In other embodiments, a tumor may be HER2⁺ as determined by immunohistochemistry but negative for HER2 amplification as determined by FISH (i.e., HER2⁺, FISH−). Chromogenic in situ hybridization (CISH) may also be used to determine HER2 gene amplification as an alternative to FISH.

By “co-administration” is meant concurrent or sequential administration of two different therapeutic agents (a first therapeutic agent and a second therapeutic agent) where both administrations are delivered close enough in time to each other that the first and second therapeutic agents become simultaneously present in patient receiving the co-administration.

By “disease” is meant any condition or disorder that damages or interferes with the normal function of a cell, tissue, or organ.

The term “doxorubicin” refers to the drug with the chemical name (8S,10S)-10-(4-amino-5 hydroxy-6-methyl-tetrahydro-2H-pyran-2-yloxy)-6,8,11-trihydroxy-8-(2-hydroxyacetyl)-1-methoxy-7,8,9,10-tetrahydrotetracene-5,12-dione. It is marketed under the trade names Adriamycin PFS®, Adriamycin RDF®, or Rubex®. Doxorubicin, like all anthracyclines, it is believed to work by intercalating DNA and thereby interfering with DNA replication and/or repair. Typically, the drug is administered intravenously, e.g., in the form of the hydrochloride salt. Doxorubicin is photosensitive, and containers comprising it should be covered by an aluminum bag or other opaque container to prevent light from damaging it.

“Subject” or “patient” refers to a human patient.

“Therapeutic agent” means a drug. A therapeutic agent may decrease, suppress, attenuate, diminish, arrest, or stabilize the development or progression of disease, disorder, or infection in a eukaryotic host organism.

“Therapeutically effective amount” refers to an amount of a therapeutic agent that provides a desired biological, therapeutic, and/or prophylactic result. That result may be reduction, amelioration, palliation, lessening, delaying, and/or alleviation of one or more of the signs, symptoms, or causes of a disease, or any other desired alteration of a biological system. In reference to cancers (e.g., HER2 overexpressing cancers), a therapeutically effective amount comprises an amount sufficient to cause a tumor to shrink and/or to decrease the growth rate of the tumor (e.g., to suppress tumor growth), or to prevent or delay other unwanted cell proliferation. In some embodiments, a therapeutically effective amount is an amount sufficient to delay tumor development. In some embodiments, a therapeutically effective amount is an amount sufficient to prevent or delay tumor recurrence. A therapeutically effective amount may be administered in one or more administrations. The therapeutically effective amount of a drug or composition may: (i) reduce the number of cancer cells; (ii) reduce tumor size; (iii) inhibit, retard, slow to some extent, and/or stop cancer cell infiltration into peripheral organs; (iv) inhibit (i.e., slow to some extent and may stop) tumor metastasis; (v) inhibit tumor growth; (vi) prevent or delay occurrence and/or recurrence of tumor; and/or (vii) relieve to some extent one or more of the symptoms associated with the cancer. In other aspects, a “therapeutically effective amount” may be an amount shown to produce a treatment outcome of CR, PR, or SD and described below:
CR (Complete Response): Disappearance of all target lesions (tumors). Any pathological lymph nodes must have reduction in short axis to <10 mm;
PR (Partial Response): At least a 30% decrease in the sum of the diameters of target lesions, taking as reference the baseline sum diameters;
SD (Stable Disease): Neither sufficient shrinkage to qualify for PR nor sufficient increase to qualify for PD (described below), taking as reference the smallest sum diameters while on treatment. (Note: a change of 20% or less that does not increase the sum of the diameters by 5 mm or more is coded as stable disease). To be assigned a status of stable disease, measurements must have met the stable disease criteria at least once after treatment is commenced at a minimum interval of 6 weeks.
PD (Progressive Disease): At least a 20% increase in the sum of the diameters of target lesions, taking as reference the smallest sum during treatment (this includes the baseline sum if that is the smallest during treatment). In addition to the relative increase of 20%, the sum must also demonstrate an absolute increase of at least 5 mm. (Note: the appearance of one or more new lesions is also considered progression).

“LABC” indicates locally advanced breast cancer.

“MBC” indicates metastatic breast cancer.

Patients can be tested or selected for one or more of the above described clinical attributes prior to, during or after treatment.

### EMBODIMENTS

Compositions and methods are provided that are effective for treating patients with histologically or cytologically confirmed advanced cancer that is positive for HER2 (i.e., is HER2⁺).

In certain embodiments, prior to initial administration of the immunoliposome, an antihistamine is administered orally or intravenously as prophylactic premedication.

In one embodiment, compositions comprising MM-302 for use in in combination with an anti-HER2 antibody, as well as methods of using the compositions for the treatment of breast cancer, are provided. In other embodiments, the invention provides methods for co-administering MM-302 with trastuzumab and compositions comprising MM-302 for administration in combination with trastuzumab, wherein the combination is administered (or is for administration) according to a particular clinical dosage regimen (i.e., at a particular dose amount and according to a specific dosing schedule).

In some embodiments, the cancer is a HER2+ solid tumor, e.g., a melanoma, a cholangiocarcinoma, clear cell sarcoma, esophageal, head and neck, endometrial, prostate, breast, ovarian, gastric, gastro-esophageal junction (GEJ), colon, colorectal, lung, bladder, pancreatic, salivary gland, liver, skin, brain or renal tumor. In other embodiments, the cancer is squamous cell cancer, small-cell lung cancer, non-small cell lung cancer, cervical cancer, or thyroid cancer. In certain embodiments the breast cancer is an early stage breast cancer. In other embodiments the breast cancer is histologically or cytologically characterized as invasive cancer of the breast. In yet another embodiment, the breast cancer is either or both of locally advanced and metastatic.

In an additional embodiment, the breast cancer is not amenable to resection with curative intent; e.g., the cancer tumor(s) is (are) inoperable.

### MM-302 Liposomes

“MM-302” refers to a HER2-targeted immunoliposome comprising an anthracycline anti-cancer therapeutic. Immunoliposomes are antibody (typically antibody fragment) targeted liposomes that provide advantages over non-immunoliposomal preparations because they are selectively internalized by cells bearing cell surface antigens targeted by the antibody. Such antibodies and immunoliposomes are described, for example, in the following US patents and patent applications: U.S. Pat. Nos. 7,871,620, 6,214,388, 7,135,177, and 7,507,407 (“Immunoliposomes that optimize internalization into target cells”); U.S. Pat. No. 6,210,707 (“Methods of forming protein-linked lipidic microparticles and compositions thereof”); U.S. Pat. No. 7,022,336 (“Methods for attaching protein to lipidic microparticles with high efficiency”); and U.S. Pat. Nos. 7,892,554 and 7,244,826 (“Internalizing ErbB2 antibodies.”). Immunoliposomes targeting HER2 can be prepared in accordance with the foregoing patent disclosures. Such HER2 targeted immunoliposomes include MM-302, which comprises the F5 anti-HER2 antibody fragment and contains doxorubicin. MM-302 contains an average of 45 copies of mammalian-derived F5-scFv (anti-HER2) per liposome.

An MM-302 liposome is a unilamellar lipid bilayer vesicle of approximately 75-110 nm in diameter that encapsulates an aqueous space that contains doxorubicin. The lipid membrane is composed of phosphatidylcholine, cholesterol, and a polyethyleneglycol-derivatized phosphatidylethanolamine in the amount of approximately one PEG molecule for 200 phospholipid molecules, of which approximately one PEG chain for each 1780 phospholipid molecules bears at its end an F5 single-chain Fv antibody fragment that binds immunospecifically to HER2.

Preferred tumors for treatment with MM-302 are those in which the tumor cells overexpress HER2. A tumor that overexpresses HER2 is one that is identified as being HER2³⁺ or HER2²HercepTest™, or HER2 FISH+ by fluorescence in situ hybridization. In some embodiments, MM-302 may be administered to a patient having a tumor that is HER2¹⁺ but which is also FISH+. Alternatively, MM-302 may be administered to a patient having a tumor that is FISH negative but is scored as HER2³⁺ or HER2²⁺ by IHC. Alternatively, a preferred tumor that overexpresses HER2 is one that expresses an average of 200,000 or more receptors per cell, as quantified by the methods described in the Examples.

In certain embodiments, MM-302 is co-administered with trastuzumab in the doses described below. In the MM-302 context, “mg/m²” indicates mg of doxorubicin (formulated as MM-302) per square meter of body surface area of the patient. In certain embodiments, MM-302 is administered (as a monotherapy or in a combination therapy regimen) to a patient that has not previously been treated with an anthracycline therapeutic (an “anthracycline naïve” patient).

In various embodiments, MM-302 is administered in combination with another HER2-targeted monoclonal antibody, e.g., pertuzumab, T-DM1 or MM-111 (as disclosed, e.g., in U.S. Pat. No. 8,927,695 and Publication Nos. 20110059076, and 20140017264).

### Dosage and Administration of MM-302

MM-302 is administered by IV (intravenous) infusion at, e.g., 30 mg/m² on day 1 of each 21-day cycle. In another embodiment, MM-302 is administered at 40 mg/m2 on day 1 of each 21- or 28-day cycle. Prior to administration, the appropriate dose of MM-302 must be diluted in 5% Dextrose Injection, USP. Care should be taken not to use in-line filters or any bacteriostatic agents such as benzyl alcohol.

In other embodiments, including monotherapy embodiments, MM-302 is administered at a dose that ranges from about 1 m g/m² to about 100 mg/m².

Pretreatment with or concomitant use of anti-emetics may be considered according to institutional guidelines. The actual dose of MM-302 to be administered is determined by calculating the patient's body surface area at the beginning of each treatment cycle. A ±5% variance in the calculated total dose is permitted for ease of dose administration. MM-302 drug solution should be inspected for particulate matter, discoloration, and cloudiness prior to administration. MM-302 drug solution that is discolored, cloudy or has a noticeable insoluble precipitate should not be administered.

### Dose Modification for Cardiac Systolic Dysfunction

Cardiac function is assessed by either echocardiogram (ECHO) or multi gated acquisition radionuclide angiography (MUGA) at baseline and throughout treatment. At baseline all patients must have a left ventricular ejection fraction (LVEF) of ≧50%. Throughout the course of treatment, any patient who develops symptomatic (CTCAE Grade 2 or higher) congestive heart failure (CHF) will discontinue study treatment.

At each cardiac assessment, if LVEF is ≧50% than the patient can continue treatment and future cardiac assessments as defined. Likewise, if a patient has an LVEF of ≧46% to 49% and a <15 absolute percentage point drop from baseline, that patient can continue treatment and future cardiac assessments as defined.

If a patient has an LVEF of ≧46% to 49% and a ≧15 absolute percentage point drop from baseline, than treatment will be withheld and a repeat LVEF assessment will be performed 3 weeks later. Likewise if a patient has an LVEF of ≦45% than treatment will be withheld and a repeat LVEF assessment will be performed 3 weeks later.

At the repeat assessment, if LVEF is either ≧50% or ≧46% to 49% and <15 absolute percentage point drop from baseline, the patient can resume study treatment. If at repeat assessment the LVEF is either ≦45% or ≧46% to 49% and 15 absolute percentage points from baseline, the patient should discontinue study treatment.

### Doses, Preparation and Administration of Trastuzumab

Preparation of trastuzumab should be followed as stated in the package insert. In one embodiment, the initial weekly dose of trastuzumab may range from about 50 mg/kg to about 2 mg/kg. For example, in one embodiment, the initial weekly dose of trastuzumab is 4 mg/kg as a 90 minute infusion followed by subsequent weekly doses of 2 mg/kg as 30 minute IV infusions. In another embodiment, the initial weekly dose of trastuzumab is 6 mg/kg as a 90 minute infusion followed by subsequent weekly doses of 4 mg/kg as 30 minute IV infusions. In another embodiment, the initial weekly dose of trastuzumab is 8 mg/kg as a 90 minute infusion followed by subsequent weekly doses of 2, 4, or 6 mg/kg as 30 minute IV infusions.

### MM-302 In Vitro Pharmacology

In vitro pre-clinical pharmacology studies of MM-302 have demonstrated that MM-302 cross-reacts with cynomolgus HER2 but not with rat HER2. Additionally, the number of HER2 receptors needed per cell to optimize the binding of MM-302 is approximately 200,000 HER2 receptors per cell. Below this level, binding is low and comparable with untargeted pegylated liposomal doxorubicin. Above this level, binding dramatically increases with smaller increases in receptor number. MM-302 does not effectively bind to or enter human cardiomyocytes. The level of MM-302 uptake into human cardiomyocytes is on the same order as untargeted pegylated liposomal doxorubicin. In contrast, the uptake of free doxorubicin is relatively much higher compared to both MM-302 and untargeted pegylated liposomal doxorubicin. The primary mechanism of action of MM-302 is the use of the HER2 protein to selectively deliver doxorubicin into tumor cells that express greater than approximately 200,000 HER2 receptors per cell.

### Pharmaceutical Compositions

Pharmaceutical compositions suitable for administration to a patient are preferably in liquid form for intravenous administration.

In general, compositions typically comprise a pharmaceutically acceptable carrier. As used herein, the term “pharmaceutically acceptable” means approved by a government regulatory agency listed in the U.S. Pharmacopeia or another generally recognized pharmacopeia for use in animals, particularly in humans. The term “carrier” refers to a diluent, adjuvant, excipient, or vehicle with which the therapeutic agent is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline and aqueous dextrose and glycerol solutions may be employed as carriers, particularly for injectable solutions). Liquid compositions for parenteral administration can be formulated for administration by injection or continuous infusion. Routes of administration by injection or infusion include intravenous, intraperitoneal, intramuscular, intrathecal, and subcutaneous. In one embodiment, both MM-302 and an anti-HER2 antibody are administered intravenously (e.g., separately or together over the course of a predetermined period of time, e.g., one hour).

MM-302 for intravenous infusion (e.g., over the course of one hour) is supplied as a clear liquid solution in sterile, single-use vials containing 10.1 ml of MM-302 at a concentration of 25 mg/ml in 20 mM histidine, 150 mM sodium chloride, pH 6.5, which should be stored at 2-8° C.

Doxorubicin is supplied in the hydrochloride form as a sterile red-orange lyophilized powder containing lactose and as a sterile parenteral, isotonic solution with sodium chloride and is also supplied as a sterile red-orange aqueous solution containing sodium chloride 0.9%.

Doxorubicin is for IV use only.
Doxorubicin has the following structural formula:

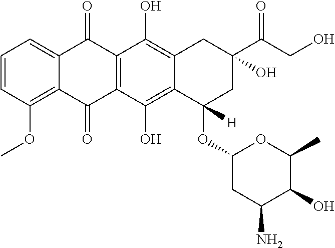 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1]

### Combination Therapy

According to the techniques herein, anti-HER2 antibodies or other anti-HER2 therapeutics may be administered as follow-on treatment in combination with MM-302 in order to effect improvement in subjects having breast cancer. In one embodiment, the anti-HER2 antibody is trastuzumab. Exemplary aspects of administration are set for in the Examples below. Other, alternate aspects of administration are set forth in the nine immediately following paragraphs.

As used herein, adjunctive or combined administration (co-administration) may include simultaneous administration of the therapeutic agents in the same or different dosage form, or separate administration of the therapeutic agents (e.g., sequential administration of MM-302 and trastuzumab). For example, an additional therapeutic antibody (e.g., trastuzumab) may be simultaneously administered with MM-302, wherein both the additional therapeutic antibody and MM-302 are formulated together. Alternatively, an additional therapeutic antibody can be administered in combination with the MM-302, wherein both the additional therapeutic antibody and MM-302 are formulated for separate administration and are administered concurrently or sequentially. For example, MM-302 may be administered first, followed by the administration of the anti-HER2 therapeutic antibody. Alternatively, the additional therapeutic antibody may be administered first, followed by administration of MM-302. Such concurrent or sequential co-administration preferably results in both MM-302 and trastuzumab being simultaneously present in treated patients.

In another embodiment, an anti-HER2 antibody may be formulated for intravenous administration. In particular embodiments, the additional therapeutic antibody may be administered at a dose that ranges from about 100 mg/kg to about 1 mg/kg. In other embodiments, the therapeutic anti-HER2 antibody may be administered at a dose that ranges from about 50 mg/kg to about 2 mg/kg. In other embodiments, the additional therapeutic anti-HER2 antibody may be administered at a dose that ranges from about 40 mg/kg to about 3.22 mg/kg. In still other embodiments, the additional therapeutic anti-HER2 antibody may be administered as a dose of 40 mg/kg, 35 mg/kg, 30 mg/kg, 25 mg/kg, 20 mg/kg, 15 mg/kg, 12 mg/kg, 10 mg/kg, 8 mg/kg, 6 mg/kg, 4 mg/kg, and/or 3.2 mg/kg. In one embodiment, the dose of additional therapeutic antibody may be varied over time. For example, the additional therapeutic antibody may be initially administered at a high dose and may be lowered over time. In another embodiment, the additional therapeutic antibody is initially administered at a low dose and increased over time. In another embodiment, a dose of 40 mg/kg of anti-HER2 antibody may be administered once per week for two weeks, followed by a dose of 20 mg/kg of an additional therapeutic anti-HER2 antibody in combination with MM-302.

### Treatment Protocols

Suitable follow-on treatment protocols are set forth in the Examples below. Alternate protocols include, for example, those wherein (A) the MM-302 may be administered to a patient (i.e., a human subject) once per every three weeks, e.g., over a course of up to fourteen three-week cycles (at a dose of 30-50 mg/m² per cycle), and (B) the doxorubicin free anti-cancer therapeutic is administered to a patient at least once per every three weeks over the same course of up to fourteen three-week cycles.

In an alternate embodiment, the doxorubicin-free anti-cancer therapeutic (e.g., trastuzumab) may be administered in combination with an amount of MM-302 at an interval measured of at least seven days. A suitable weekly dosage of trastuzumab is 2 mg/kg.

In one embodiment, the first dose of the MM-302 and/or the signaling-inhibitory anti-HER2 antibody is a loading dose, i.e., a dose that is larger than the dose given in subsequent administrations (as such, the maintenance dose).

In another embodiment, MM-302 is administered once every three weeks or once every four weeks. The administration cycle may be repeated, as necessary.

In another embodiment, the amount of doxorubicin-free anti-cancer therapeutic administered may be constant for each dose. In another embodiment, the amount of antibody administered may vary with each dose. For example, a maintenance dose of the antibody may be higher than, or the same as, the loading dose that is first administered. In another embodiment, the maintenance dose of the antibody can be lower than the loading dose.

In one follow-on treatment embodiment, an anti-HER2 antibody may be administered as a monotherapy prior to at least one cycle of anti-HER2 antibody/MM-302 combination therapy. In one embodiment, anti-HER2 antibody monotherapy may be administered for two weeks, wherein the anti-HER2 antibody may be administered at 6 mg/kg the first week and at 4 mg/kg the second week.

In one follow-on treatment embodiment, MM-302 may be administered as a monotherapy prior to at least one cycle of anti-HER2 antibody/MM-302 combination therapy. In one embodiment, the MM-302 monotherapy may be administered every four weeks, wherein the MM-302 may be administered at 30 mg/m², 40 mg/m², or 50 mg/m² once every four weeks.

The following Examples are merely illustrative and should not be construed as limiting the scope of this disclosure in any way as many variations and equivalents will become apparent to those skilled in the art upon reading the present disclosure.

### EXAMPLES

### Example 1

### Gastric Cancer Xenograft Model

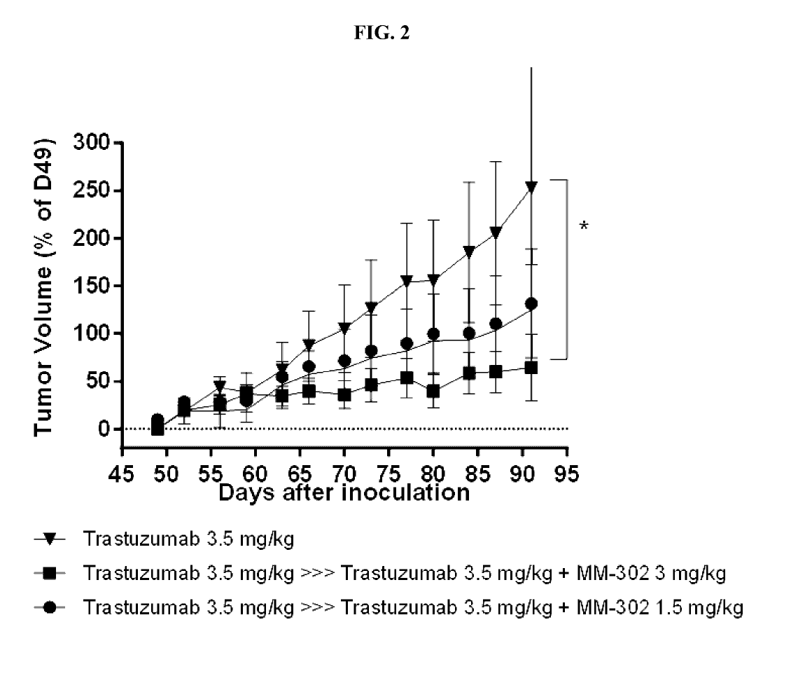

The response to the combination of MM-302 and trastuzumab after failure of response to trastuzumab was evaluated in a gastric cancer xenograft model (NCI-N87). Mice were inoculated with NCI-N87 gastric cancer cells (ATCC® CRL-5822™) at 10×10⁶; into the right flank. When tumor volumes reached about 250 mm³, mice were treated with trastuzumab at a loading dose of 7 mg/kg, followed by a dose of 3.5 mg/kg q3d. Upon a 50% increase in tumor volume during treatment with trastuzumab, indicating failure of response, i.e., resistance, to trastuzumab, mice were randomized into three treatment groups that received one of the following treatments: a) trastuzumab at 3.5 mg/kg q3d and MM-302 at 3 mg/kg q3d (squares); b) trastuzumab at 3.5 mg/kg q3d and MM-302 at 1.5 mg/kg q3d (circles); or c) they were maintained on the trastuzumab schedule (3.5 mg/kg q3d, triangles). For the first two groups, MM-302 was dosed q1w for a total of 3 doses (day 49, 56, and 63). Changes in tumor volume as well as in mouse weight were monitored twice a week. As shown in FIG. 2, at day 91, the treatment with trastuzumab and MM-302 (3 mg/kg) showed a significantly higher tumor growth inhibition compared to the trastuzumab single agent treatment (2-way ANOVA; *). Treatment with the MM-302 1.5 mg/kg dose (circles) in combination with trastuzumab also demonstrated increased tumor growth inhibition compared to the trastuzumab single agent treatment.

### Example 2

### Breast Cancer Xenograft Model

The response to the MM-302 and trastuzumab combination after failure to respond to T-DM1 was evaluated in the BT-474-M3 breast cancer xenograft model.

To obtain the BT-474-M3 HER2-overexpressing human breast cancer cell line, BT-474 cells (ATCC® HTB-20™) were passaged twice through mice with the fastest growing two tumors out of ten selected for ex-vivo propagation during each round of selection. Tumors were excised and cultured ex-vivo to obtain the M3 sub-line that was verified by SNP analysis. (see Noble, Cancer Chemother. Pharmacol. 2009 64:741-51).

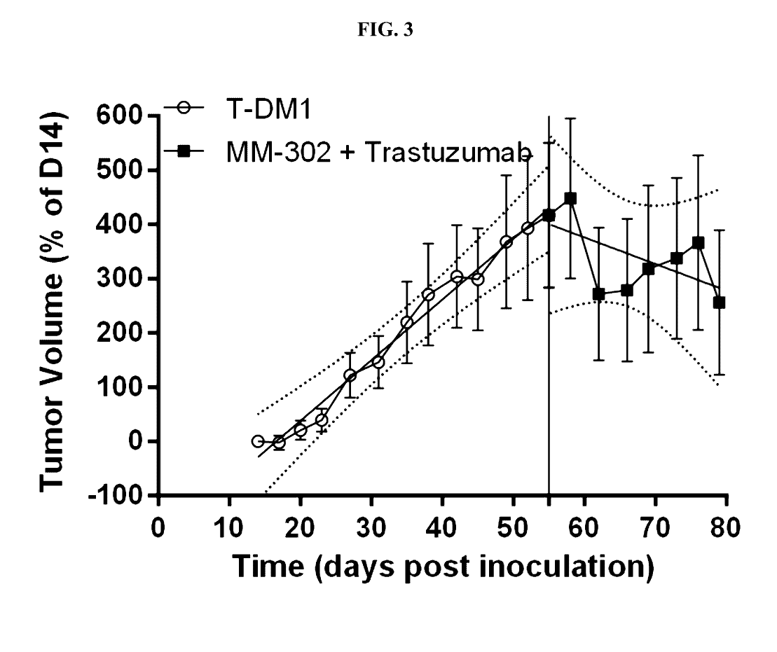

Mice were inoculated with BT-474-M3 breast cancer cells (15×10⁶; into the mammary fat pad). When tumor volumes reached about 360 mm³, mice were treated with T-DM1 at either 1, 2, or 4 mg/kg (q1w) (white circles). The tumors failed to respond to single agent T-DM1. At day 55, the mice were switched from T-DM1 treatment to the combination of MM-302 (3 mg/kg, q1w for a total of 3 doses; day 55, 62 and 69) and trastuzumab (at a loading dose of 8 mg/kg followed by 6 mg/kg q1w) (black squares). Changes in tumor volume as well as in mouse weight were monitored twice a week. Changes in tumor volume relative to the start of treatment with T-DM1 (day 14) are shown in FIG. 3. The change of treatment at day 55 (indicated by change in data points from open circles to black squares) resulted in a better control of tumor growth, as indicated by the surprisingly significant difference between the slopes of the curve for days 0-55 (positive slope) during T-DM1 treatment as compared to days 55-80 (negative slope) during MM-302 and trastuzumab treatment (linear regression analysis).

### Example 3

### Clinical Parameters

### Patient Population

In a preferred embodiment, each patient to be treated in accordance with this invention will have locally advanced/metastatic HER2-positive breast cancer and will have received prior treatment with trastuzumab and have progressed on or been intolerant to each of pertuzumab and ado-trastuzumab emtansine, preferably in the LABC/MBC setting. Patients who received pertuzumab and ado-trastuzumab in the neoadjuvant/adjuvant setting are not excluded; however, they must have also received pertuzumab and ado-trastuzumab in the LABC/MBC setting and have shown to be resistant and/or intolerant to each of these anti-cancer therapies (in any setting).

### Disease-Specific Treatment Criteria

In certain embodiments, each patient to be treated in accordance with this invention must have histologically or cytologically confirmed invasive cancer of the breast.

In certain embodiments, each breast cancer patient to be treated in accordance with this invention must have documented locally advanced/metastatic disease (i.e., LABC/MBC) defined by the physician, which is not amenable to resection with curative intent

In certain embodiments, each patient to be treated in accordance with this invention must have HER2-positive breast cancer, e.g., as defined by ASCO/CAP 2013 guidelines (as per Wolff, et al., J. Clin. Oncol. 2013: 31 (31) 3997-4013).

In certain embodiments, each patient to be treated in accordance with this invention must have archived tissue available to submit for analysis or be willing to undergo a biopsy for HER2 evaluation.

In certain embodiments, each patient to be treated in accordance with this invention must not have a medical condition for which systemic cancer chemotherapy is contraindicated.

In certain embodiments, each patient to be treated in accordance with this invention must have documented disease progression (via RECIST or clinical progression) or intolerance during or after the most recent treatment for LABC/MBC

In certain embodiments, each patient to be treated in accordance with this invention must have progressed on, or be intolerant to treatment with these therapies:
a. pertuzumab in the LABC/MBC setting, and/or
b. ado-trastuzumab emtansine in the LABC/MBC setting.

In certain embodiments, each patient to be treated in accordance with this invention must have been previously treated with trastuzumab (in any setting—which may have been previously administered with or without pertuzumab)

In certain embodiments, each patient to be treated in accordance with this invention will have Eastern Cooperative Oncology Group (ECOG) Performance Status (PS) of 0 or 1.

In certain embodiments, patients may be treated who have central nervous system (CNS) metastases if they have been previously treated for CNS metastases and become stable without symptoms for 4 weeks after completion of treatment and remain stable and are off steroids for at least 4 weeks prior to treatment in accordance with this invention.

### Hematologic, Biochemical and Organ Function

In certain embodiments, each patient to be treated in accordance with this invention must have adequate bone marrow reserves as evidenced by:
a. Absolute neutrophil count (ANC) ≧1,500/μL
b. Platelet count ≧100,000/μL
c. Hemoglobin ≧9 g/dL (transfusions allowed)

In certain embodiments, each patient to be treated in accordance with this invention must have adequate coagulation function as evidenced by International normalized ratio (INR) and activated partial thromboplastin time (aPTT)≦1.5 Upper Limit of Normal (ULN; unless on therapeutic coagulants).

In certain embodiments, each patient to be treated in accordance with this invention must have adequate hepatic function as evidenced by:
a. Serum total bilirubin within normal limits;
b. Aspartate aminotransferase (AST), Alanine aminotransferase (ALT) up to 3× upper limit of normal; and
c. Serum Albumin ≧2.5 g/dL.

Each patient to be treated in accordance with this invention must have adequate renal function as evidenced by a serum creatinine ≦1.5× upper limit of normal

In certain embodiments, each patient to be treated in accordance with this invention must have adequate cardiac function as evidenced by a measured left ventricular ejection fraction of ≧50% by MUGA or ECHO. Measurements by ECHO are preferably be read as a single value and not as a range.

In certain embodiments, each patient to be treated in accordance with this invention must be recovered to at least CTCAE (v4.0) grade 1 from any clinically relevant toxic effects of any prior surgery, radiotherapy or other therapy intended for the treatment of breast cancer. For peripheral neuropathy, up to CTCAE (v4.0) Grade 2 is acceptable for a patient with a pre-existing condition. A patient with any grade of alopecia and/or fatigue may be enrolled.

### Exclusion Criteria

### Disease Specific Exclusion Criteria:

In certain embodiments, no patient is to be treated in accordance with this invention who has previously been treated with doxorubicin, liposomal doxorubicin, epirubicin, mitoxantrone or any other anthracycline derivative (i.e., is anthracycline-naïve) (except optionally valrubicin or other anthracyclines that have not been systemically administered).

In certain embodiments, no patient is to be treated in accordance with this invention who has known hypersensitivity to any of the components of MM-302 or who have had hypersensitivity reactions to fully humanized monoclonal antibodies.

In certain embodiments, no patient is to be treated in accordance with this invention who has a history of intolerance to trastuzumab (i.e. a grade 3 or 4 infusion reaction). (Patients with a history of mild infusion reaction to trastuzumab who have previously been successfully re-challenged after an infusion reaction with or without prophylactic medication may be treated in accordance with this invention)

### Cardiac Exclusion Criteria:

In certain embodiments, no patient is to be treated in accordance with this invention with any class of NYHA congestive heart failure (CHF) or heart failure with preserved ejection fraction (HFPEF).

In certain embodiments, no patient is to be treated in accordance with this invention with hypertension (systolic BP >150 mm Hg or diastolic BP >100 mm Hg) that is not controlled by adequate standard anti-hypertensive treatment

In certain embodiments, no patient is to be treated in accordance with this invention with known unstable angina pectoris

In certain embodiments, no patient is to be treated in accordance with this invention with a known history of serious cardiac arrhythmias requiring treatment (exception: atrial fibrillation and paroxysmal supraventricular tachycardia)

In certain embodiments, no patient is to be treated in accordance with this invention with a prolonged QTc interval (≧450 ms)

In certain embodiments, no patient is to be treated in accordance with this invention who previously discontinued trastuzumab due to unacceptable cardiac toxicity or infusion related reactions

In certain embodiments, no patient is to be treated in accordance with this invention with a history of LVEF decline to below 50% during or after prior trastuzumab/lapatinib or other HER2 directed therapy

In certain embodiments, no patient is to be treated in accordance with this invention with current dyspnea at rest due to complications of advanced malignancy or other disease that requires continuous oxygen therapy

### General Exclusion Criteria:

No patient is to be treated in accordance with this invention who is pregnant or breast feeding

In certain embodiments, no patient is to be treated in accordance with this invention with an active infection or with an unexplained fever >38.5° C. during screening visits or on the first scheduled day of dosing (at the discretion of the physician, Each patient to be treated in accordance with this invention with tumor fever may be enrolled)

In certain embodiments, no patient is to be treated in accordance with this invention with a history of allogeneic transplant.

### Example 4

### Treatment Parameters

### MM-302 Formulation, Packaging, and Labeling

MM-302 drug product is formulated in sterile 10 mM L-histidine-HCl as a buffer (pH 6.5), and 10% sucrose to maintain isotonicity.

MM-302 is supplied in single use vials containing 10 mL of MM-302 at a concentration of 2 mg/ml. The vials contain a 5% excess to facilitate the withdrawal of the label amount from each 10 ml vial. MM-302 drug product is reddish in color and is sterile dispensed into 10 mL clear glass vials closed with crimped rubber caps at 10 mL (20 mg doxorubicin hydrochloride) per vial.

MM-302 is packaged and labeled according to country specific guidelines.

### MM-302 Product Storage and Stability

Upon receipt of MM-302, vials must be refrigerated at 2° C. to 8° C. (36° F. to 46° F.) until use. Vials should not be frozen or shaken. MM-302 must be stored in original carton to protect from light. If a temperature deviation from the allowed 2° C.-8° C. is found either during shipment or storage, contact the Sponsor to determine if the drug is still appropriate for use. MM-302 is not to be used beyond the expiration date provided.

### MM-302 Dosage, Premedication, Preparation and Administration

MM-302 will be dosed at 30 mg/m² for all patients randomized to the experimental arm on day 1 of each 21 day cycle.

The appropriate dose of MM-302 must be diluted in 5% Dextrose Injection, USP. DO NOT USE in-line filters or any bacteriostatic agents such as benzyl alcohol.

The actual dose of MM-302 to be administered will be determined by calculating the patient's body surface area at the beginning of each cycle. A ±5% variance in the calculated total dose will be allowed for ease of dose administration.

Body surface area (BSA) will be calculated using the DuBois formula or equivalent (e.g., the Mosteller formula) as follows: BSA (m²)=0.007184× [height (cm)]0.725× [weight (kg)]0.425.

As applicable, each drug solution should be inspected for particulate matter, discoloration, and cloudiness prior to administration. If the solution is discolored, cloudy or if an insoluble precipitate is noted, the drug solution should not be used.

All patients receiving MM-302 will receive prophylactic premedication with 25-50 mg diphenhydramine or equivalent antihistamine. The premedication may be administered PO (by mouth) or IV prior to dosing MM-302. Patients who tolerate initial doses of MM-302 (C1D1 and C2D1) without infusion reactions then may not require premedication in subsequent cycles, per the physician's judgment. Patients whose infusion reactions are manifest by myalgia or other pain symptoms should also receive acetaminophen 650 mg PO with the antihistamine therapy. In general, MM-302 is considered to have low emetogenic potential. Pretreatment with or concomitant use of anti-emetics may be considered according to institutional guidelines.

MM-302 is administered by IV infusion over 60 minutes on the first day of each 21 day cycle. The first cycle Day 1 is a fixed day. Subsequent doses should be administered on the first day of each cycle ±3 days.

Prior to administration, the appropriate dose of MM-302 is diluted in 5% Dextrose Injection, USP.

### Management of Toxicities Related to MM-302

The following guidelines for the management of toxicities are designed to maximize treatment for patients who appear to be benefiting from treatment while ensuring patient safety.

Patients should be carefully monitored for toxicity. Adverse reactions, such as PPE, hematologic toxicities, and stomatitis may be managed by dose delays and adjustments. Treatment may be delayed for up to 42 days for toxicities. If the adverse event(s) does not return to baseline or <Grade 1 within 42 days from the last administered dose, drug treatment must be discontinued.

A maximum of two dose reductions per patient will be permitted in response to either hematologic or non-hematologic toxicities. Subjects who require a third dose reduction of 25% due to toxicity will be discontinued from treatment with MM-302.

Patients who discontinue MM-302 for toxicity can remain on continued trastuzumab treatment if felt to be in the best interest of the patient as determined by the physician.

Following the first appearance of a Grade 2 or higher adverse reactions, the dosing should be adjusted or delayed as described in the following tables. Once the dose has been reduced, it should not be increased at a later time.

### Dose Adjustments for Hepatic Toxicity:

For patients with impaired hepatic function in the absence of progressive disease, the MM-302 dosage will be reduced if the bilirubin is elevated as follows:

TABLE 2
MM-302 Dose Adjustments for Hepatic Toxicity

| Total Billirubin | Percentage of Starting |
|---|---|
| (mg/dL) | Dose of MM-302 |
| <1.2 | 100% |
| 1.2-3.0 | 50% |
| >3.0 | 25% |

### Dose Adjustments for Palmar-Plantar Erythrodysesthesia Syndrom (Ppe) and Stomatitis:

For patients with PPE and/or stomatitis, the following dose delays and adjustments will be followed. As stated above, a maximum of two dose reductions per patient is allowed. If a third dose reduction is required, the patient must discontinue MM-302.

TABLE 3
MM-302 Dose Adjustments for Palmar-Plantar
Erythrodysesthesia Syndrom (PPE) and Stomatitis
Only 2 dose reductions are allowed. Patients who require
a third dose reduction will discontinue MM-302 treatment

| NCI | | |
|---|---|---|
| Toxicity | Dose Adjustment for | Dose Adjustment for |
| Grade | PPE | Stomatitis |
| 1 | No change unless patient has | No change unless patient has |
| | experienced previous Grade 3 | experienced previous Grade 3 |
| | PPE. If so, delay dose to wait | or 4 stomatitis If so, delay |
| | for toxicity to resolve and | dose to wait for toxicity to |
| | decrease dose by 25%. | resolve and decrease dose by |
| | | 25%. |
| 2 | Delay dosing until resolved | Delay dosing until resolved |
| | grade 0-1. | grade 0-1. |
| | If resolved to grade 0-1 within | If resolved to grade 0-1 within |
| | 42 days from last infusion and | 42 days from last infusion and |
| | there are no prior grade 2-3 | there are no prior grade 2-4 |
| | PPE, continue treatment at | stomatitis, continue treatment |
| | previous dose. If patient | at previous dose. If patient |
| | experienced previous grade 2- | experienced previous grade 2- |
| | 3 toxicity, continue treatment | 4 toxicity, continue treatment |
| | at 25% dose reduction | at 25% dose reduction |
| 3 | Delay dosing until resolved | Delay dosing until resolved |
| | to grade 0-1. | to grade 0-1. |
| | Decrease dose by 25%. If | Decrease dose by 25%. If |
| | there is no resolution within | there is no resolution within |
| | 42 days from last infusion, | 42 days from last infusion, |
| | MM-302 should be | MM-302 should be |
| | discontinued. | discontinued. |
| 4 | N/A as grade 4 PPE does not | Delay dosing until resolved |
| | exist per CTCAE criteria | to grade 0-1. |
| | | Decrease dose by 25%. If |
| | | there is no resolution within |
| | | 42 days from last infusion, |
| | | MM-302 should be |
| | | discontinued. |

### Dose Adjustments for Hematologic Toxicity:

For patients with ANC and/or platelet deficiencies, the following dose adjustments will be followed as outlined in Table 4 below.

TABLE 4
MM-302 Dose Adjustments for Hematologic
Toxicity on Day 1 of Each Cycle

| | ANC | Platelets | |
|---|---|---|---|
| Grade | (x 106/L) | (x 106/L) | Modification |
| 1 | 1,500-1,900 | 75,000-150,000 | Resume treatment |
| | | | with no dose reduction |
| 2 | 1,000-<1,500 | 50,000-<75,000 | Wait until ANC ≧1,500 |
| | | | and platelets ≧75,000; |
| | | | re-dose with no dose |
| | | | reduction |
| 3 | 500-999 | 25,000-<50,000 | Wait until ANC ≧1,500 |
| | | | and platelets ≧75,000; |
| | | | re-dose with no dose |
| | | | reduction |
| 4 | <500 | <25,000 | Wait until ANC ≧1,500 |
| | | | and platelets ≧75,000; |
| | | | re-dose at 25% dose |
| | | | reduction |

### Management of Infusion Reactions to MM-302

All patients receiving MM-302 will receive prophylactic premedication with 25-50 mg diphenhydramine or equivalent antihistamine. The premedication may be administered orally or IV prior to dosing MM-302. Patients who tolerate initial doses of MM-302 (C1D1 or C2D1) without infusion reactions then may not require premedication in subsequent cycles, per the physician's judgment. Patients whose infusion reactions are manifest by myalgia or other pain symptoms should also receive acetaminophen 650 mg PO with the antihistamine therapy.

Infusion reactions will be defined according to the National Cancer Institute CTCAE (Version 4.0) definition of an allergic reaction/infusion reaction and anaphylaxis as defined below. Institutional guidelines may be used instead of the guidelines provided below:
Grade 1: Mild transient reaction; infusion interruption not indicated; intervention not indicated
Grade 2: Therapy or infusion interruption indicated but responds promptly to symptomatic treatment (e.g., antihistamines, NSAIDS, narcotics, IV fluids); prophylactic medications indicated for <24 hours
Grade 3: Prolonged (e.g., not rapidly responsive to symptomatic medication and/or brief interruption of infusion); recurrence of symptoms following initial improvement; hospitalization indicated for clinical sequelae
Grade 4: Life-threatening consequences; urgent intervention indicated

### Treatment Guidelines for the Management of Mm-302 Infusion Reactions:

### Grade 1

Slow infusion rate by 50%
Monitor patient every 15 minutes for worsening of condition

### Grade 2

Stop infusion
Administer diphenhydramine hydrochloride 50 mg IV, acetaminophen 650 mg orally, and oxygen
Resume infusion at 50% of the prior rate once infusion reaction has resolved
Monitor patient every 15 minutes for worsening of condition
For all subsequent infusions, pre-medicate with diphenhydramine hydrochloride 25-50 mg IV

### Grade 3

Stop infusion and disconnect infusion tubing from patient
Administer diphenhydramine hydrochloride 50 mg IV, dexamethasone 10 mg IV, bronchodilators for bronchospasm, and other medications or oxygen as medically necessary
Patient should be discontinued MM-302 if:
   the infusion reaction lasts more than 24 hours, or
   the patient cannot receive the entire dose due to the infusion reaction, or
   a grade 3 infusion reaction occurs in spite of pre-medication
Patients who continue on treatment:
   Resume infusion at 50% of the prior rate once infusion reaction has resolved
   Monitor patient every 15 minutes for worsening of condition
   Pre-medicate with diphenhydramine hydrochloride 25-50 mg IV for all subsequent infusions

### Grade 4

Stop the infusion and disconnect infusion tubing from patient
Administer epinephrine, bronchodilators or oxygen as indicated for bronchospasm
Administer diphenhydramine hydrochloride 50 mg IV, dexamethasone 10 mg IV
Consider hospital admission for observation
Patient should be discontinued from treatment

For patients who experience a Grade 1, Grade 2, or Grade 3 infusion reaction, at the discretion of the treating physician, future infusions may be administered at a reduced rate (e.g., over 90 minutes). For patients who experience a recurrence of any of these infusion reactions, administer dexamethasone 10 mg IV and all subsequent infusions are to be pre-medicated with diphenhydramine hydrochloride 50 mg IV, dexamethasone 10 mg IV, and acetaminophen 650 mg orally.

### Trastuzumab Dosage and Administration

Trastuzumab is dosed following the infusion of MM-302.

Trastuzumab will be administered Q3W as an IV loading dose of 8 mg/kg for Cycle 1, and 6 mg/kg maintenance dose for subsequent cycles.

The dose of trastuzumab does not need to be recalculated unless the body weight has changed by more than ±10% from baseline.

A loading dose may not be required for patients who have recently been treated with trastuzumab. Patients who have been treated on a Q3W regimen within the last 4 weeks or Q1W regiment within the last 2 weeks should begin dosing at the maintenance dose of 6 mg/kg.

### Management of Toxicities Due to Trastuzumab

Trastuzumab is administered until physician-assessed radiographic or clinical progressive disease, or unmanageable toxicity. Administration may be delayed to assess or treat adverse events such as cardiac adverse events or myelosuppression. No dose reduction should be taken.

If the patient misses a dose (or doses) of trastuzumab, a re-loading dose of trastuzumab should be given. Patients who miss one cycle or more of treatment (i.e. 6 weeks or more between infusions) should be given the re-loading dose of 8 mg/kg at the following treatment.

Trastuzumab may be held for a maximum of 42 days from last administered dose. Patients who require longer dose delays shall have their dose discontinued. On the basis of the physician's judgment, patients who discontinue trastuzumab may continue treatment with MM-302 or discontinue treatment altogether.

### Management of Cardiac Toxicity

Patients should have a baseline LVEF ≧50%. LVEF should be monitored regularly. If a physician is concerned that an adverse event may be related to cardiac dysfunction, an additional LVEF measurement should be performed. Any patient who develops clinical signs and symptoms suggesting congestive heart failure, with the diagnosis confirmed by a suggestive chest X-ray and a drop in LVEF by MUGA or ECHO should have treatment discontinued. CHF should be treated and monitored according to standard medical practice.

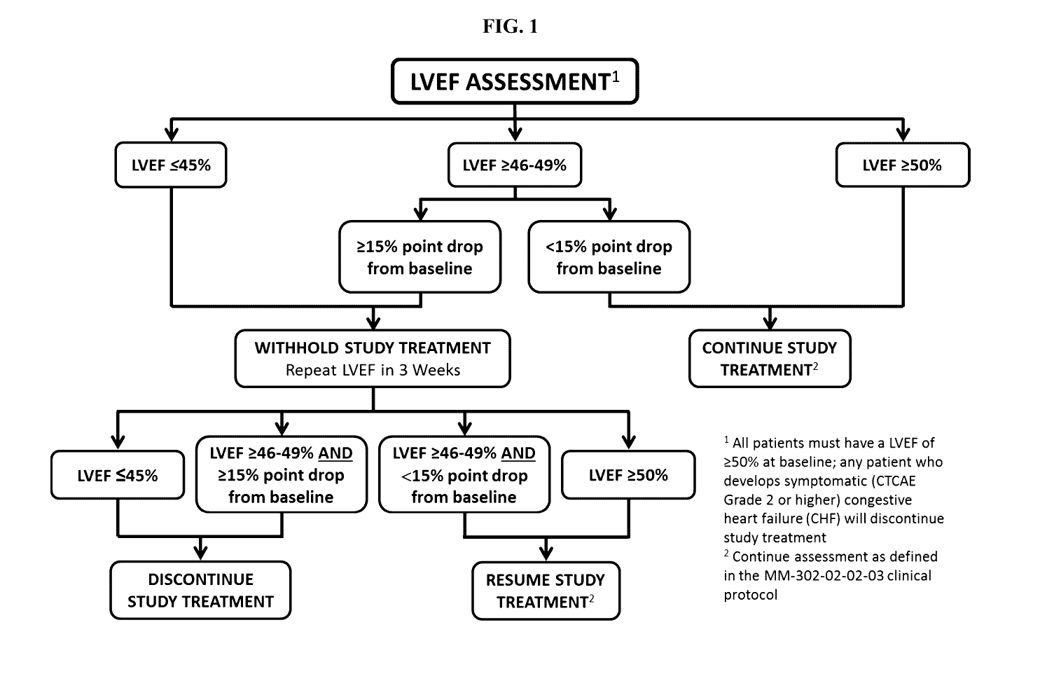

Patients with changes in LVEF are to be treated according to the algorithm set forth in FIG. 1. Patients who develop symptomatic (CTCAE Grade 2 or higher) congestive heart failure should be discontinued from treatment.

### Concomitant and Excluded Therapies

All inter-current medical conditions and complications of the underlying malignancy are to be treated at the discretion of the treating physician according to acceptable local standards of medical care. Patients may receive analgesics, anti-emetics, anti-diarrheal antibiotics, anti-pyretics, hematopoietic growth factors, and blood products as necessary. For patients with bone metastases, standard of care treatments such as bisphosphonates and denosumab may be used.

### Example 5

### HER2 Therapy-Resistant Patients Respond to Treatment with MM-302+Trastuzumab

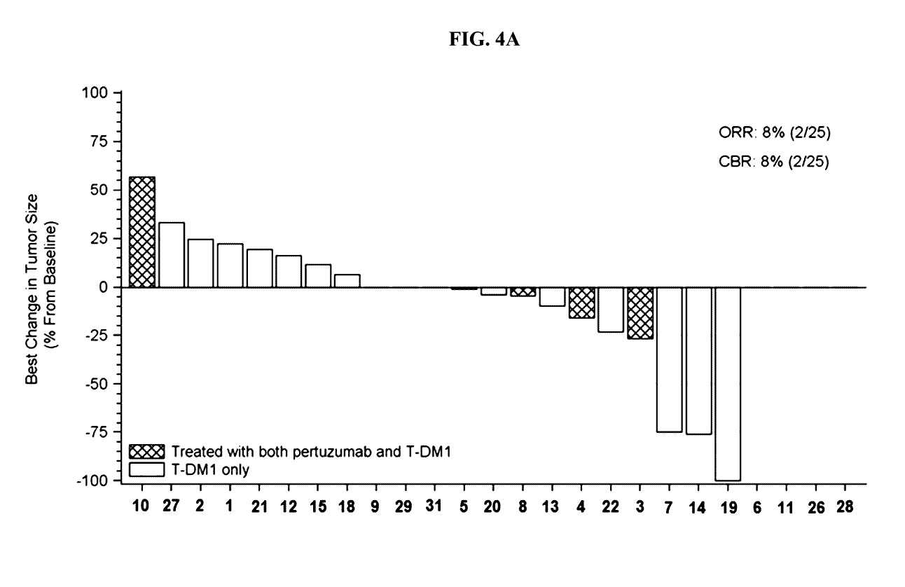

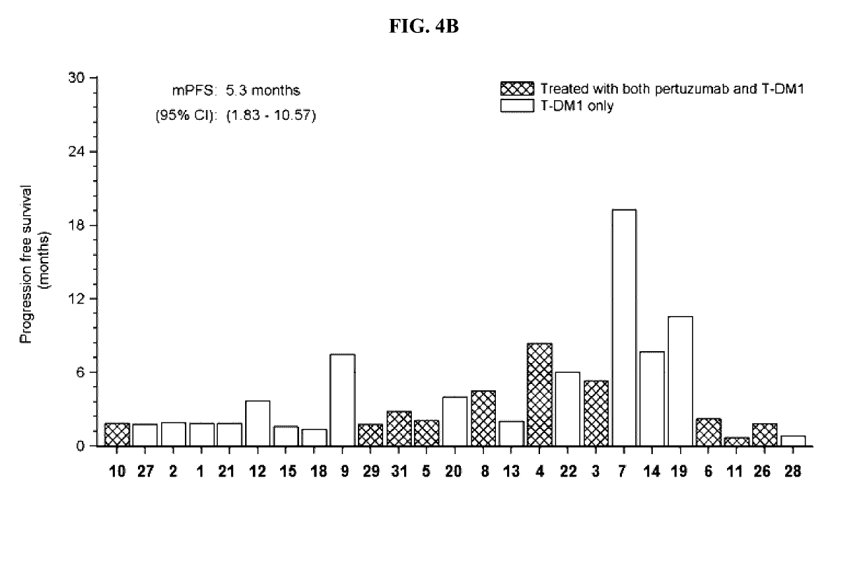

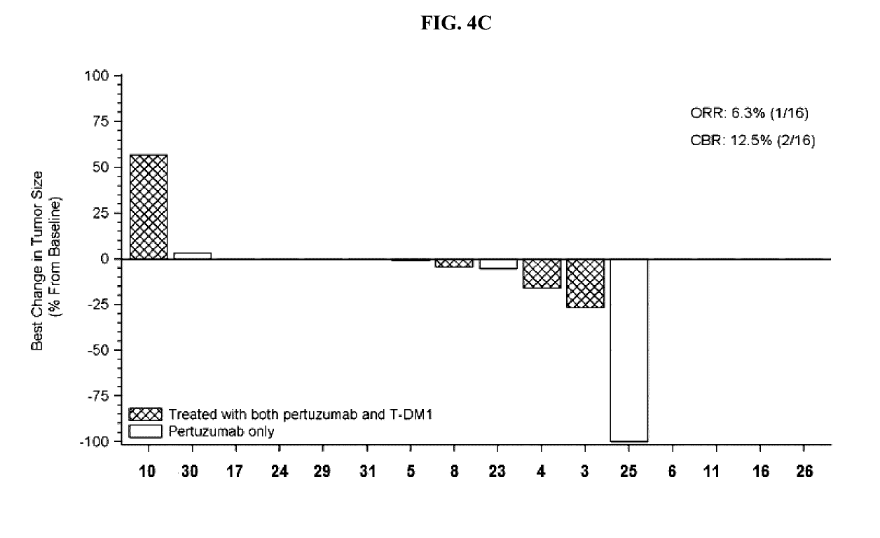

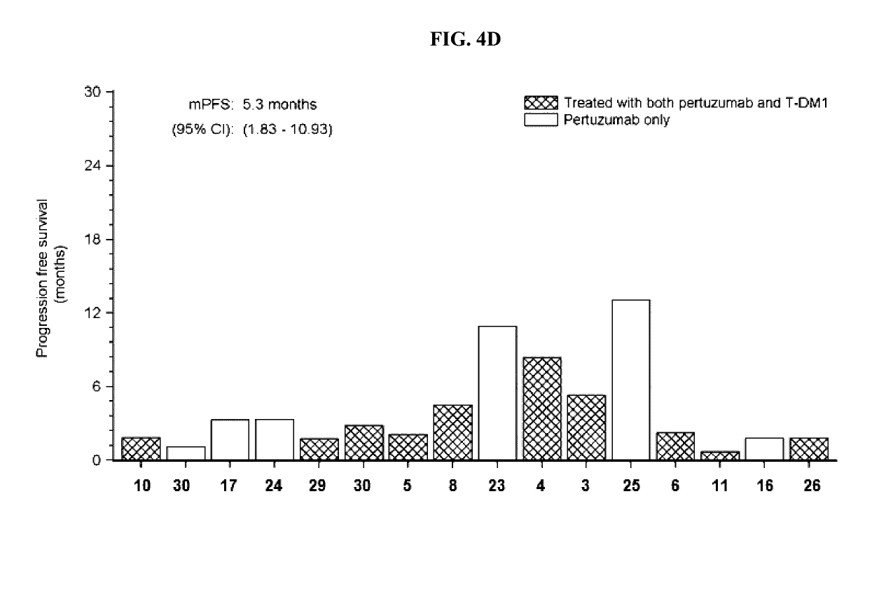

Patients who had progressed on treatment with T-DM1 (FIGS. 4A and 4B) or pertuzumab (FIGS. 4C and 4D) received MM-302 doses of ≧30 mg/m² in combination with trastuzumab with or without cyclophosphamide (See Table 5 below). Patients who had previously received and progressed on treatment with both T-DM1 and pertuzumab are indicated by hatched bars (FIGS. 4A-4D). FIGS. 4A and 4C show the change in patients' tumor size after treatment with MM-302 and trastuzumab. FIGS. 4B and 4D show patient progression-free survival in months. Together the data show that treatment with the combination of MM-302 and trastuzumab can extend PFS for patients that have progressed on an anti-HER2 therapeutic, and that the combination treatment resulted in a reduction in tumor size for many of the patients.

TABLE 5
Treatment Regimens for Patient Data in FIG. 4

| Patient | |
|---|---|
| Number | Treatment |
| 1 | 30 mg/m² MM-302, q4w + 4 mg/kg trastuzumab, q2w |
| 2 | 30 mg/m² MM-302, q4w + 4 mg/kg trastuzumab, q2w |
| 3 | 30 mg/m² MM-302, q3w + 6 mg/kg trastuzumab, q3w |
| 4 | 30 mg/m² MM-302, q3w + 6 mg/kg trastuzumab, q3w |
| 5 | 30 mg/m² MM-302, q3w + 6 mg/kg trastuzumab, q3w |
| 6 | 30 mg/m² MM-302, q3w + 6 mg/kg trastuzumab, q3w + |
| | 450 mg/m² cyclophosphamide, q3w |
| 7 | 30 mg/m² MM-302, q4w + 4 mg/kg trastuzumab, q2w |
| 8 | 30 mg/m² MM-302, q4w + 4 mg/kg trastuzumab, q2w |
| 9 | 30 mg/m² MM-302, q3w + 6 mg/kg trastuzumab, q3w |
| 10 | 30 mg/m² MM-302, q3w + 6 mg/kg trastuzumab, q3w |
| 11 | 30 mg/m² MM-302, q3w + 6 mg/kg trastuzumab, q3w |
| 12 | 30 mg/m² MM-302, q3w + 6 mg/kg trastuzumab, q3w + |
| | 450 mg/m² cyclophosphamide, q3w |
| 13 | 30 mg/m² MM-302, q3w + 6 mg/kg trastuzumab, q3w + |
| | 450 mg/m² cyclophosphamide, q3w |
| 14 | 30 mg/m² MM-302, q3w + 6 mg/kg trastuzumab, q3w + |
| | 450 mg/m² cyclophosphamide, q3w |
| 15 | 30 mg/m² MM-302, q3w + 6 mg/kg trastuzumab, q3w |
| 16 | 30 mg/m² MM-302, q3w + 6 mg/kg trastuzumab, q3w + |
| | 450 mg/m² cyclophosphamide, q3w |
| 17 | 30 mg/m² MM-302, q4w + 4 mg/kg trastuzumab, q2w |
| 18 | 30 mg/m² MM-302, q3w + 6 mg/kg trastuzumab, q3w |
| 19 | 30 mg/m² MM-302, q3w + 6 mg/kg trastuzumab, q3w + |
| | 450 mg/m² cyclophosphamide, q3w |
| 20 | 30 mg/m² MM-302, q3w + 6 mg/kg trastuzumab, q3w + |
| | 450 mg/m² cyclophosphamide, q3w |
| 21 | 30 mg/m² MM-302, q3w + 6 mg/kg trastuzumab, q3w + |
| | 450 mg/m² cyclophosphamide, q3w |
| 22 | 30 mg/m² MM-302, q3w + 6 mg/kg trastuzumab, q3w + |
| | 450 mg/m² cyclophosphamide, q3w |
| 23 | 40 mg/m² MM-302, q4w + 4 mg/kg trastuzumab, q2w |
| 24 | 40 mg/m² MM-302, q4w + 4 mg/kg trastuzumab, q2w |
| 25 | 40 mg/m² MM-302, q4w + 4 mg/kg trastuzumab, q2w |
| 26 | 30 mg/m² MM-302, q3w + 6 mg/kg trastuzumab, q3w |
| 27 | 30 mg/m² MM-302, q3w + 6 mg/kg trastuzumab, q3w |
| 28 | 30 mg/m² MM-302, q3w + 6 mg/kg trastuzumab, q3w + |
| | 450 mg/m² cyclophosphamide, q3w |
| 29 | 30 mg/m² MM-302, q3w + 6 mg/kg trastuzumab, q3w |
| 30 | 30 mg/m² MM-302, q3w + 6 mg/kg trastuzumab, q3w |
| 31 | 30 mg/m² MM-302, q3w + 6 mg/kg trastuzumab, q3w + |
| | 450 mg/m² cyclophosphamide, q3w |

### Example 6

### The Combination of MM-302 and T-DM1 in HER2-Positive Cell Lines

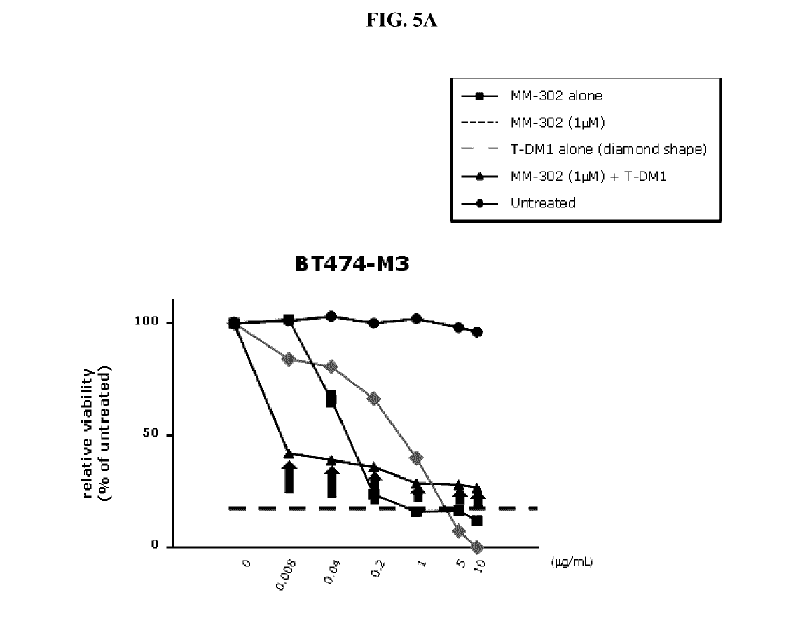

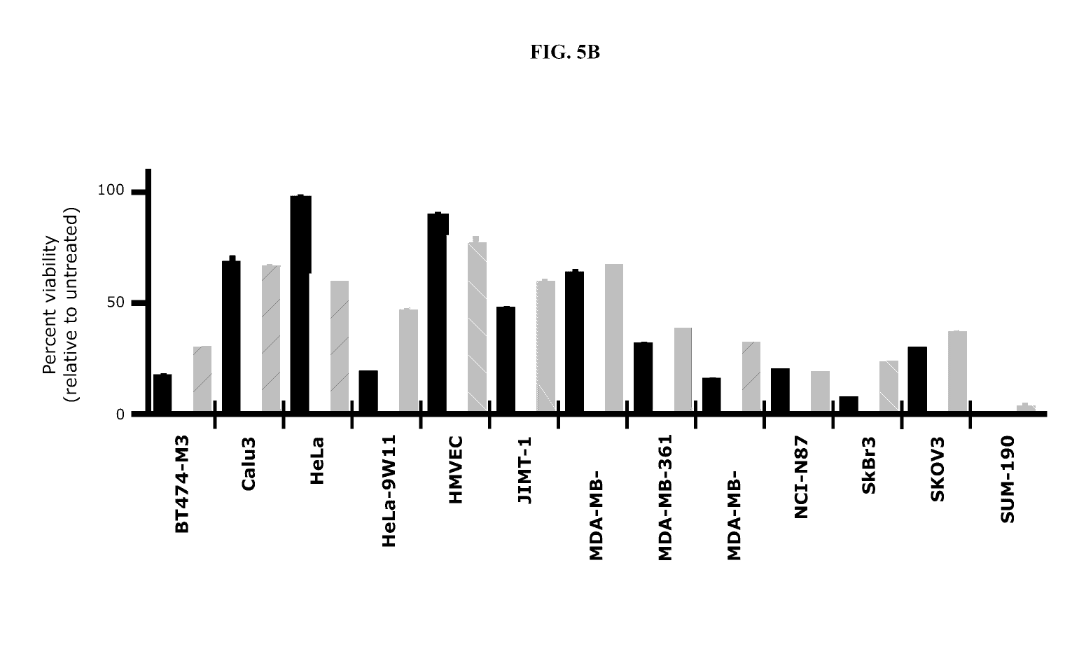

The MM-302+T-DM1 combination was tested in vitro against a number of cell lines. HER2-expressing cell lines were treated with a range of doses of MM-302 alone from 0 to 10 μg/ml, T-DM1 alone at a range from 0 to 10 μg/ml, or the combination of MM-302 (at 1 μM) and T-DM1 at a range from 0 to 10 μg/ml (added simultaneously). Cell viability was assessed using the PrestoBlue® cell viability reagent (Life Technologies) at 96 hours relative to an untreated control. FIG. 5A shows viability data from BT-474-M3 cells that were untreated (circles), treated with MM-302 alone (squares), T-DM1 alone (diamonds) or MM-302 plus T-DM1 (triangles). The dashed line corresponds to the level of cancer cell viability when treated with MM-302 at 1 μM. A beneficial combinatorial effect of the MM-302+T-DM1 combination should result in a reduction in the level of cancer cell viability to a level below this dashed line; however, this reduction was not observed in most cell lines tested, including the cell lines HeLa-9w11. This was true for most cell lines tested, including the cell lines HeLa-9W11, MDA-MB-175, MDA-MB-361, MDA-MB-453, JIMT-1, NCI-N87, SkBr3, SKOV3, and SUM190. In contrast, the combination showed some benefit toward reducing cell viability in other cell lines such as Calu-3, HeLa, and HMVEC cells. FIG. 5B shows a results summary of the cell lines tested, with black bars representing MM-302 at 1 μM, light gray bars representing T-DM1 at 1 μg/mL, and dark gray bars representing the combination of MM-302 at 1 μM and T-DM1 at 1 μg/mL.

### EQUIVALENTS

Those skilled in the art will recognize, or be able to ascertain and implement using no more than routine experimentation, many equivalents of the specific embodiments described herein. Such equivalents are intended to be encompassed by the following claims. Any combination, or combinations, of the embodiments disclosed in the dependent claims are contemplated to be within the scope of the disclosure.

### INCORPORATION BY REFERENCE

The disclosure of each and every U.S. and foreign patent and pending patent application and publication referred to herein is specifically incorporated by reference herein in its entirety.

What is claimed is:

1. A method of follow-on treatment of a HER2-positive tumor in a human patient comprising administering to the patient a therapeutically effective amount of an immunoliposome comprising encapsulated doxorubicin and a HER2-binding antibody, in combination with an anti-HER2 therapeutic that comprises trastuzumab or another anti-HER2 antibody, wherein, prior to the follow-on treatment, the patient had received treatment with one or more of 1) trastuzumab, 2) pertuzumab, and 3) ado-trastuzumab emtansine, and had experienced tumor progression following such prior treatment initiation with, or was intolerant to treatment with pertuzumab and had experienced tumor progression following such prior treatment initiation with, or was intolerant to such prior treatment with ado-trastuzumab emtansine.

2. The method of claim 1, wherein (i) the immunoliposome is an immunoliposome comprising encapsulated doxorubicin and an immunoliposome-associated anti-HER2 antibody that is not an inhibitor of HER2 signaling and is oriented on the immunoliposome so as to be able to bind to an antigen that is external to the immunoliposome and (ii) the anti-HER2 therapeutic is a doxorubicin-free therapeutic comprising an anti-HER2 antibody that a) is an inhibitor of HER2 signaling, or that b) does not bind to the same epitope of HER2 that is bound by the immunoliposome-associated antibody, or that c) does not compete with the immunoliposome-associated antibody for immunospecific binding to HER2, or that a) and b), or that a) and c), or that b) and c), or that a) and b) and c).

3. The method of claim 2, wherein the immunoliposome is administered intravenously.

4. The method of claim 3, wherein the patient has not previously been treated with a systemically administered anthracycline.

5. The method of claim 4, wherein the immunoliposome-associated anti-HER2 antibody is a single-chain Fv (scFv).

6. The method of claim 5, wherein scFv is an F5 scFv comprising the amino acid sequence encoded by ATCC plasmid deposit designation PTA7843.

7. The method of claim 6, wherein the immunoliposome is MM-302 and the doxorubicin-free anti-cancer therapeutic is trastuzumab.

8. The method of claim 7, wherein the tumor is a breast cancer tumor.

9. The method of claim 8, wherein the breast cancer is histologically or cytologically characterized as invasive cancer of the breast.

10. The method of claim 9, wherein the breast cancer is either or both of locally advanced and metastatic.

11. The method of claim 10, wherein the breast cancer is not amenable to resection with curative intent.

12. The method of claim 11, wherein, prior to initial administration of the immunoliposome, an antihistamine is administered orally or intravenously as prophylactic premedication.

13. The method of claim 12, wherein the method comprises at least one 3-week treatment cycle in which the signaling-inhibitory anti-HER2 antibody is administered at a dose of 6 mg/kg per administration and the immunoliposome is administered at a dose of 30 mg/m² per administration;
and wherein,
when the at least one cycle is a single cycle, the anti-HER2 antibody and the immunoliposome are each administered once;
and wherein, when the at least one cycle is a plurality of cycles, the signaling-inhibitory anti-HER2 antibody is administered every three weeks and the immunoliposome is administered every three weeks or every four weeks.
